(19) United States
(12) Patent Application Publication
(10) Pub. No.: US 2019/0209028 A1
(43) Pub. Date: Jul. 11, 2019

(54) DEVICES AND METHODS FOR SENSING BIOLOGIC FUNCTION

(71) Applicant: INTEL CORPORATION, Santa Clara, CA (US)

(72) Inventor: Amit Sudhir Baxi, Bangalore (IN)

(73) Assignee: INTEL CORPORATION, Santa Clara, CA (US)

(21) Appl. No.: 16/305,778

(22) PCT Filed: Jun. 30, 2016
(86) PCT No.: PCT/US2016/040476

§ 371 Date: Nov. 29, 2018

(51) Int. Cl. A61B 5/021; A61B 5/0408; A61B 5/00

(52) U.S. Cl. CPC A61B 5/02108; A61B 5/0408; A61B 5/0006; A61B 5/6833; A61B 2562/0215; A61B 5/0017

(57) ABSTRACT

Discussed generally herein are methods and devices for sensing biological function using a flexible, stretchable patch. In one or more embodiments, a device can include a stretchable, flexible first substrate material mechanically coupled to and on the stretchable, flexible fabric, and first metallization mechanically coupled to and on the first substrate material, the first metallization including first pads, meandering traces, and at least one of electrocardiogram (ECG) electrodes, a stretch sensor, and at least one stretch limiting patch.

### TECHNICAL FIELD

This disclosure relates generally to sensing one or more biological functions. One or more embodiments regard, more specifically, determining blood pressure without using a cuff.

### BACKGROUND ART

According to a 2015 study by the Center for Disease Control of the United States, about twenty-nine percent of American adults experience high blood pressure (e.g., hypertension). That is about seventy million American adults. Monitoring blood pressure is usually measured using a cuff wrapped around an arm or a wrist. The air cuff is inflated to occlude arterial blood flow. The cuff is then slowly deflated to release air from the inflated air cuff. The pressure of air in the cuff at which the blood flow begins again is known as the systolic blood pressure (SBP). The pressure of air in the cuff at which the cuff is no longer deflating is known as the diastolic blood pressure (DBP). More formally, SBP is a measure of pressure in arteries when the heart is contracted and DBP is a measure of pressure in arteries when the heart is resting.

### BRIEF DESCRIPTION OF THE DRAWINGS

FIG. 1 illustrates, by way of example, a diagram of a PTT measurement system in use on a patient.

FIG. 2 illustrates, by way of example, a graph of ECG and PPG versus time.

FIG. 3 illustrates, by way of example, a perspective view diagram of an embodiment of a PTT measurement system.

FIG. 4 illustrates, by way of example, another perspective view diagram of the embodiment of the sensor patch system of FIG. 3.

FIG. 5 illustrates, by way of example, a perspective view diagram of an embodiment of another sensor patch system with the flex circuit attached to the stretchable, flexible patch.

FIG. 6 illustrates, by way of example, a perspective view diagram of the embodiment of the sensor patch system of FIG. 5 from an external view.

FIG. 7 illustrates, by way of example, a cross-section diagram of the system as cut along the line labelled “FIG. 7” in FIG. 6.

FIG. 8 illustrates, by way of example, a perspective view diagram of an embodiment of another sensor patch system in which the stretchable, flexible patch is situated over the sensor node.

FIG. 9 illustrates, by way of example, an exploded view diagram of a stackup of the system of FIG. 8.

FIG. 10 illustrates, by way of example, a cross-section diagram of an embodiment of another sensor patch system in which the sensors contact the skin through one or more holes in the stretchable flexible patch.

FIG. 11 illustrates, by way of example, an exploded view diagram of a stackup of the system of the FIG. 10.

FIG. 12 illustrates, by way of example, a logical circuit diagram of sensor circuitry.

FIG. 13 illustrates, by way of example, a perspective view diagram of an embodiment of an entity wearing a sensor patch system discussed herein.

FIG. 14 illustrates, by way of example, a block diagram of an embodiment of an electronic device.

### DESCRIPTION OF EMBODIMENTS

The following description and the drawings sufficiently illustrate embodiments to enable those skilled in the art to practice them. Other embodiments can incorporate structural, logical, electrical, process, or other changes. Portions and features of some embodiments can be included in, or substituted for, those of other embodiments. Embodiments set forth in the claims encompass all available equivalents of those claims.

Hypertension is more common than diabetes. Current blood pressure (BP) monitoring techniques are not portable or comfortable for continuous monitoring in ambulatory conditions. BP varies based on a variety of conditions including mobility state (stationary, awake, sleeping, walking, running, standing, sitting, exercising, or the like), calorie and type of calorie intake, hydration level, stress level, and medication ingestion, among others. As many of these conditions vary throughout a day, it can be beneficial to monitor BP throughout a given day.

It is possible to measure BP based on electrocardiogram (ECG) and photo-plethysmograph (PPG) signals. ECG is generally measured using one or more electrodes. PPG is generally measured using one or more optical sensors. A pulse transit time (PTT) is a time interval between an R-peak of the ECG signal to a corresponding, temporally correlated, peak on the PPG signal measured elsewhere on the body (typically on the finger or the wrist). PTT is inversely correlated to BP.

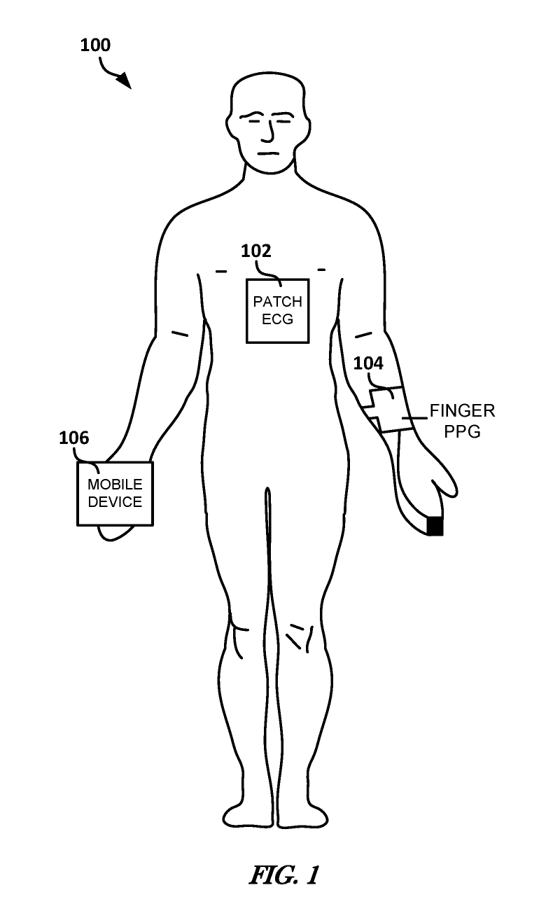

FIG. 1 illustrates, by way of example, a diagram of a PTT measurement system 100 in use on a patient. The PTT measurement system 100 includes an ECG device 102, a PPG device 104, and a mobile device 106. The ECG device 102 as illustrated is a patch device that measures ECG. The ECG is a measure of electrical activity of the heart over a period of time. Electrodes (e.g., electrically conductive elements) of the ECG device conduct this electrical activity to circuitry that convert the electrical activity to a digital number. The PPG device 104 is a device that mounts to a finger of the patient and measures PPG. The PPG device 104 can be a pulse oximeter. A pulse oximeter illuminates skin and measures changes in light reflected back to a light sensor of the pulse oximeter. Blood in the arteries causes the arteries to expand and contract as blood from the heart is transported to and away from the arteries, respectively. An expanded artery in proximity to the light sensor will reflect more light back to the sensor than the same artery that is not expanded. This difference in reflected light can be used to determine blood flow conditions in the arteries.

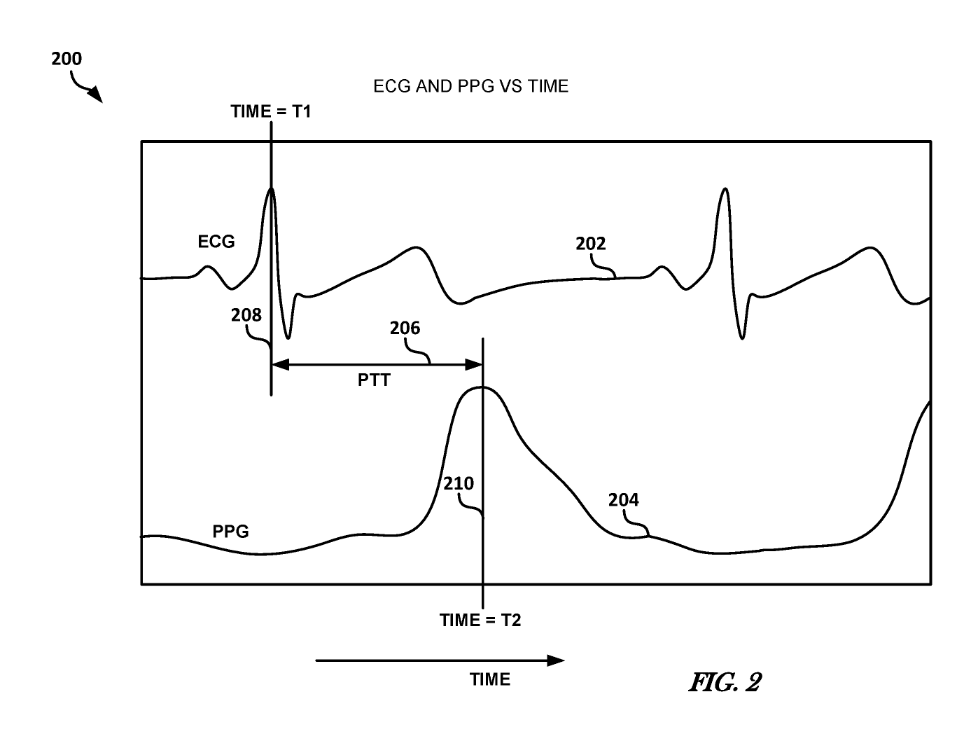

FIG. 2 illustrates, by way of example, a graph 200 of ECG and PPG versus time. The line 202 corresponds to the ECG waveform and the line 204 corresponds to the PPG waveform. The PTT 206 is measured between a peak of the ECG waveform and a corresponding peak of the PPG waveform. The peak of the ECG waveform occurs at time T1, the location corresponding to line 208, in the example of FIG. 2. The peak of the PPG waveform occurs at time T2, the location corresponding to the line 210, in the example of FIG. 2.

To get an accurate measure of the PTT 206 (and ultimately an accurate BP measurement), data from the ECG device 202 and data from the PPG device 104 need to be synchronized to within about a half millisecond of each other. If such error ranges are met, the overall error of the PTT calculation is not over a millisecond. To achieve this maximum range of error in the time synchronization of the ECG data and the PPG data, the devices can be hardwired to a shared clock. Such a setup can be quite obtrusive with wires running over the surface of the body restricting movement and creating a potential snag hazard.

In embodiments in which the ECG device 102 and the PPG device 104 wirelessly transmit their respective bio-signals to an aggregator device (e.g., the mobile device 106) the time-synchronization is difficult to achieve. This difficulty can arise at least in part because of clock skew and/or a non-deterministic delay introduced by the wireless network. An error in a PTT measurement can result in an even larger error in a BP estimation.

Using a separate ECG device 102 and PPG device 104 as in the system 100 of FIG. 1 is also considered by some to be obtrusive and/or uncomfortable. This obtrusiveness/discomfort is compounded under ambulatory conditions (i.e. user movement). Also, the signal quality of the PPG device 104 can degrade under ambulatory conditions. Motion artifacts can corrupt the PPG signal (e.g., by moving the PPG device 104) making it difficult to sufficiently locate a corresponding fiducial point of the PPG data for PTT measurement.

To address one or more of the problems discussed, a system that integrates both the ECG sensors and the PPG sensors together onto a flexible, stretchable substrate. The result is a single patch which sticks to a surface, such as human or other animal skin (see FIG. 9). The patch can transmit (e.g., wired or wirelessly transmit) time-synchronized ECG and PPG data to an aggregator device (e.g., a wireless communication device, such as a smartphone) for accurate PTT and BP estimation. Alternatively, the data may be stored on a memory of the patch over a period of time and extracted later (e.g., for lower cost monitoring or to reduce the power consumption). The patch is more usable and less-obtrusive compared to wearing two rigid devices on body, such as those of the system 100 of FIG. 1. The ECG sensors and PPG sensors can be co-located on the same patch and driven by a single clock, such as to synchronize the ECG sensors and PPG sensors. A more precise PTT measurement is possible with such time synchronization, making BP more accurate, as compared to other wirelessly transmitting systems, such as that shown in FIG. 1. Wearing the sensors on the torso of a person provides better signal quality, as compared to the system 100 of FIG. 1, as there is less movement of the torso as compared to wearing ECG/PPG sensors on the hand or other limb.

Commercially available wearable patches available in the market today include the ZIO® patch from iRhythm Technologies, Inc. of San Francisco, Calif., United States, and the SEEQ™ Mobile Cardiac Telemetry (MCT) System from Medtronic, Inc. of Dublin, Ireland. These patches sense only ECG signals and do not sense PPG signals. It is difficult to integrate the optical sensors on the same patch as the ECG sensors. There are currently no commercially available flexible patches which integrate both ECG and PPG sensors together, such as to help enable continuous sensing of BP via PTT.

A flexible, stretchable patch that includes both PPG and ECG sensors (sometimes referred to as an integrated BP patch) can be better than current known solutions in the following ways: (1) the integrated patch is less obtrusive and more usable in that, rather than wearing two separate devices on the body for PTT measurement as in the previously discussed PPT measurement system 100, the user has to wear only a single patch for BP monitoring, thus making the integrated BP patch less obtrusive and more usable; (2) the ECG and PPG signals of the integrated BP patch can be sampled by, and time-synchronized to, the same master clock on the patch, since both sets of sensors are electrically connected on the patch, thus making the PTT measurement more precise than systems that synchronize to separate PPG and ECG clocks; and/or (3) measuring PPG on a torso region (e.g., chest or trunk region) enables better PPG signal quality as there is less signal degradation due to motion of the torso in ambulatory conditions as compared to the signal degradation due to motion of fingers or hand.

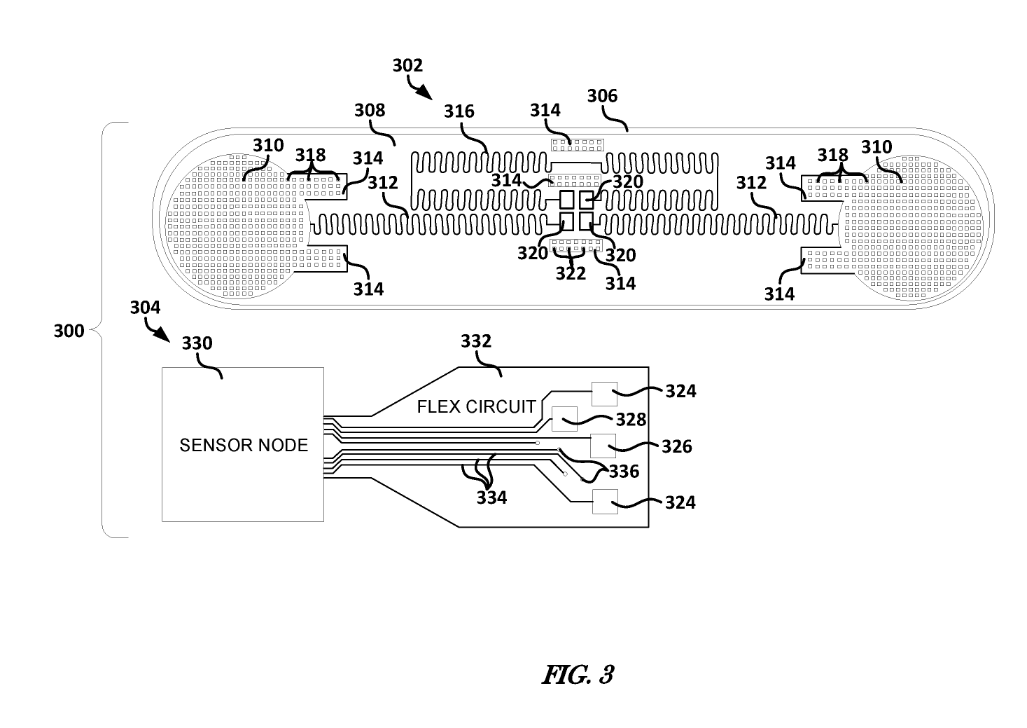

FIG. 3 illustrates, by way of example, a perspective view diagram of an embodiment of a PTT measurement system 300. The system 300 as illustrated includes a flexible, stretchable patch 302 and a control circuit 304. The perspective view provided in FIG. 3 will be referred to as a “body-side view”. The body-side view includes a view of the patch 302 as it would look from a viewer looking out from a surface of skin of an entity wearing the patch 302. The opposite view is referred to as an “external view”.

The terms “flexible” and “stretchable” as used herein are distinct terms. Flexible connotes an ability to rotate and bend. Stretchable connotes an ability to lengthen.

The patch 302 as illustrated includes an optional stretchable, flexible fabric 306, a flexible, stretchable substrate material 308 on the fabric 306, and first metallization (e.g., ECG electrode 310, meandering trace 312, stretch limiting patch 314, pad 320, or other metallization) patterned on the substrate material 308. The fabric 306 can include a material used in clothing, such as can include spandex, Lycra, vinyl, polyamide polyurethane-elastane velvet, cotton, denim, polyester, or the like. The fabric 306 provides mechanical strength for the first metallization and/or the substrate material 308, such as to help the patch 302 withstand more force in being stretched, bent, rotated, poked, or otherwise having force exerted thereon.

The substrate material 308 provides a medium that can be attached to the fabric 306 (when the fabric 306 is present). The substrate material 308 provides a medium on which the first metallization can be situated. The substrate material 308 can include a material, such as thermo-plastic polyurethane (TPU), silicone and/or polydimethylsiloxane (PDMS), poly (lactic acid) based polymers or co-polymers, other bio-compatible tri-block copolymers, thermosets (e.g., polyuria), polydimethylsiloxane (PDMS), among others. The first metallization can be situated on the substrate material 308, such as by an additive process (e.g., printing, deposition, or the like) or a subtractive process (e.g., a process that includes removing material, such as by etching). The substrate material 308 can be attached to the fabric 306 by heat pressing the substrate material 308 into the fabric 306.

The first metallization on the substrate material 308 as illustrated includes a plurality of ECG electrodes 310, meandering traces 312, stretch limiting patches 314, a stretch sensor 316, and contact pads 320 (note that not all contact pads include reference numerals so as to not obscure the view of other portions of the patch 302). The first metallization can include copper, gold, silver, aluminum, tin, titanium, palladium, platinum, nickel, a combination thereof, or other conductive material. Different metallization can include different materials. For example, the ECG electrodes 310, meandering traces 312, stretch limiting patches 314, stretch sensor 316, and/or the pads 320 can be made of different or same materials.

The ECG electrodes 310 are dry conductive electrodes, as compared to conventional ECG electrodes with jelly (that dries out within a few hours). Using jelly, the ECG signal from the ECG electrodes progressively deteriorates. The ECG electrodes 310 can include perforations 318 therein. The perforations 318 are voids in the electrodes 310. The perforations 318 aid in anchoring the electrodes 310 to the substrate 308. The perforations 318 can help reduce overall cost of the device, such as when using an additive manufacturing technique (e.g., conductive ink printing). The perforations 318 can help increase flexibility without significantly impacting performance (e.g., electrical performance characteristics) of the electrode 310.

The traces 312 electrically connect the electrodes 310 to respective pads 320. The traces 312 are formed with a meandering pattern, such as to increase the stretchability and/or flexibility of the patch 302 as compared to using generally straight traces.

The stretch limiting patches 314 can include metallization with perforations 322 similar to the perforations 318. The stretch limiting patches 314 are positioned to help prevent breakage of other metallization, such as the electrodes 310, electrical junctions between the traces 312 and other metallization, pads 320, and/or the interconnect between the fabric 306 and the substrate 308. The stretch limiting patches 314 constrain the strain in the regions in and around the stretch limiting patches and help prevent over-stretching in areas that might otherwise break if the patch 302 is over-stretched. The ECG electrodes 310 can be low-profile, thin, stretchable, flexible, low-cost, and/or disposable.

The stretch sensor 316 can provide, by way of resistance measurements, an indication of an amount to which the stretch sensor 316 is stretched. The resistance value of the meandering traces of the stretch sensor 316 can change as they are stretched more or less. This resistance value can be determined by circuitry connected to the pads 320 that are electrically connected to the stretch sensor 316. The resistance value can be interpreted, modified, or otherwise analyzed to determine a respiration rate and/or heart rate of the individual wearing the system 300.

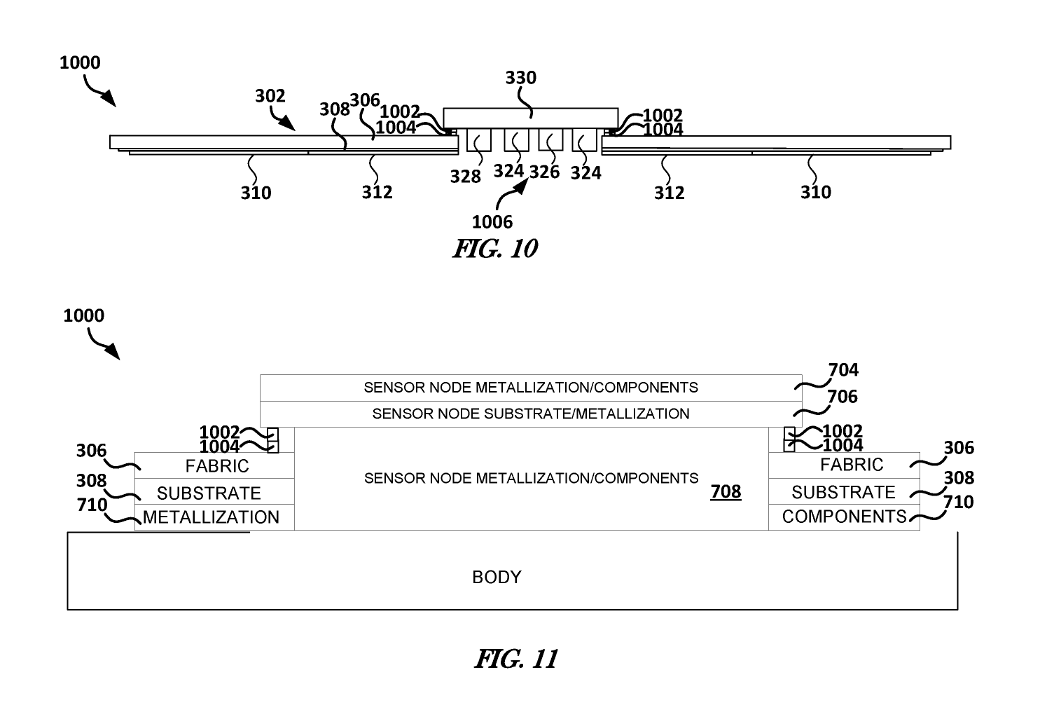

The control circuit 304 as illustrated includes a sensor node 330 and a flex circuit 332. The flex circuit 332 can be electrically and mechanically connected to the sensor node 330 by a connector (see FIG. 10).

The sensor node 330 can include electric and/or electronic components (e.g., an oscillator, resistors, transistors, processors, switches, inductors, capacitors, diodes, amplifiers, voltage and/or current regulators, voltage and/or current boosters, antennas, power sources (e.g., batteries), or the like). Circuitry of the sensor node 330 is described in more detail with regard to FIGS. 10 and 12.

The flex circuit 332 as illustrated includes a plurality of light emitting diodes (LEDs) 324, a photodiode 326, a sensor 328, traces 334, and vias 336. While not shown in this view of the flex circuit 332, the flex circuit 332 can include one or more contact pads, such as to connect the flex circuit 332 to the patch 302. The flex circuit 332, in one or more embodiments includes a polyimide substrate with second metallization (e.g., second traces 334 or other metallization) therein or thereon. The second metallization can include the second traces 334, vias, and/or second pads, such as can be used to mount the flex circuit 332 on the patch 302 or to mount the LEDs 324, photodiode 326, and/or sensor 328 on the flex circuit 332. Signals from the ECG electrodes 310 and/or the stretch sensor 316 can be provided to the sensor node 330 through second metallization on the flex circuit 332.

The LEDs 324 produce light. The light is emanated to blood of the entity wearing the system 300 and is reflected/scattered, at least partially, by the blood back to the photodiode 326. In one or more embodiments, the LEDs 324 produce green light. The photodiode 326 is capable of detecting changes in light reflected from the skin. The pulsatile blood flow changes the intensity of reflected light. Different amounts of light are reflected depending on a state of blood flow near the skin as previously discussed. In one or more embodiments, a red and an infra-red LED can be used in conjunction to help provide real-time, ambulatory monitoring of blood oxygen saturation.

The sensor 328 can include a temperature detector (TD) (e.g., a thermistor, a resistance thermometer, or a resistance TD (RTD)), a motion sensor, noise sensor, or mechanical sensor. An RTD is a component that changes resistance with temperature in a predictable manner. RTDs are typically made from a pure metal, such as copper, nickel, or platinum, wrapped in a coil around a core, such as a ceramic or glass. A motion sensor (sometimes referred to as an accelerometer) detects whether and/or a general direction of acceleration of the sensor. A noise sensor detects external electromagnetic signals and provides a signal indicative of a magnitude of the detected signals. The signals from one or more of the motion, noise, and stretch sensor can be used for adaptive noise cancellation and can help reduce motion related noise from bio-signals.

The traces 334 provide a conductive path to the sensor node 330. The vias 336 provide a path for signals to travel from one layer of the flex circuit 332 to another layer of the flex circuit 332.

Unlike current ECG-only patches available in the market today the system 300 can integrate two or more of ECG electrodes, optical PPG sensors, temperature sensor (e.g., the thermistor 328), and respiration sensor (e.g., the stretch sensor 316) onto the same flexible substrate (e.g., a combination of the flex circuit 332, substrate material 308, and the fabric 306). Mounting the rigid LEDs 324 on a flex polyimide (e.g., the substrate of the flex circuit 332) and then on substrate material 308, can help to improve reliability at rigid-flex junctions. This is at least in part due to the components being rigid and providing localized flex and stretch resistance near the components.

In one or more embodiments, the electrodes 310, meandering traces 312, and/or other metallization can be screen or inkjet printed on the substrate 308. Such as process can use a silver-based printing material (e.g., an “ink”). The LEDs 324, in one or more embodiments, can be low-profile screen printed/organic LEDs. The printing approach can be used to create low-cost, low-profile, and/or disposable patches in a volume manufacturing environment.

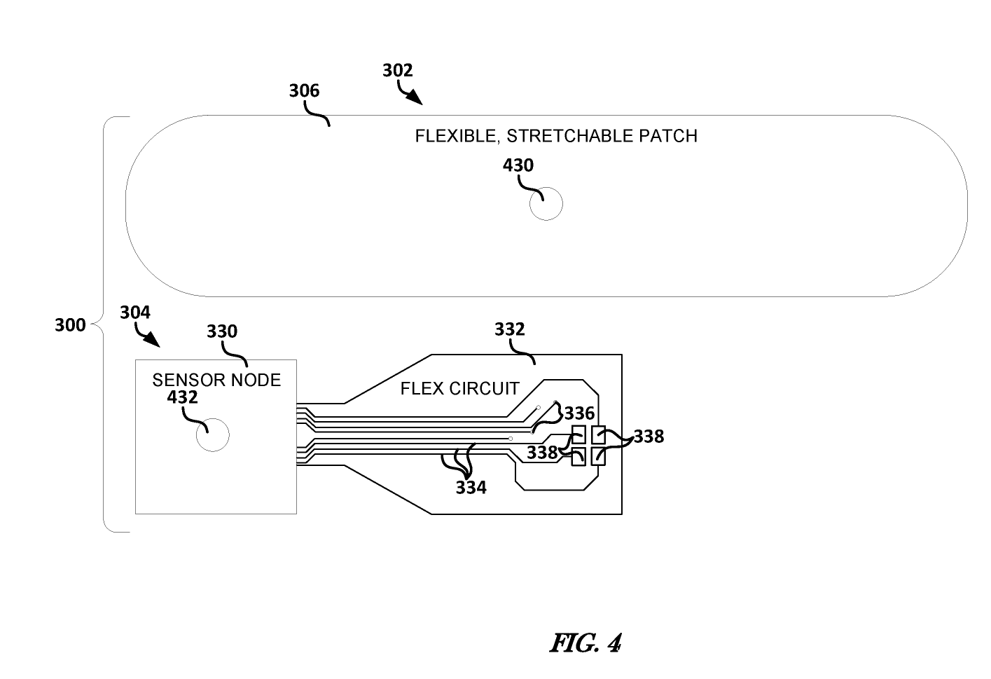

FIG. 4 illustrates, by way of example, another perspective view diagram of the embodiment of the sensor patch system 300 of FIG. 3. The view shown in FIG. 4 are the portions of the system 300 that are on opposite sides of the body side shown in FIG. 3, referred to as the external view.

The patch 302 as illustrated includes an optional male or female connection feature 430. The sensor node 330 as illustrated includes an optional mating female or male connection feature 432. The connection feature 430 can include a snap, magnet, connector, Velcro, or the like. The connection feature 432 can include the mating snap, magnet, connector, Velcro, or the like. When in contact, the connection features 430 and 432 can secure the sensor node 330 to the fabric 306. In embodiments that do not include the connection features 430 and 432, a double-sided adhesive tape, glue, or other attachment mechanism can be used to secure the sensor node 330 to the fabric 306.

The flex circuit 332 as illustrated includes contact pads 338. The contact pads 338 can be situated to align with the contact pads 320. The contact pads 338 can be in electrical and mechanical contact (e.g., by solder or a conductive adhesive) with the contact pads 320, such as to provide electrical signals from the contact pads 320 (e.g., from the electrodes 310 or stretch sensor 316) to the flex circuit 332.

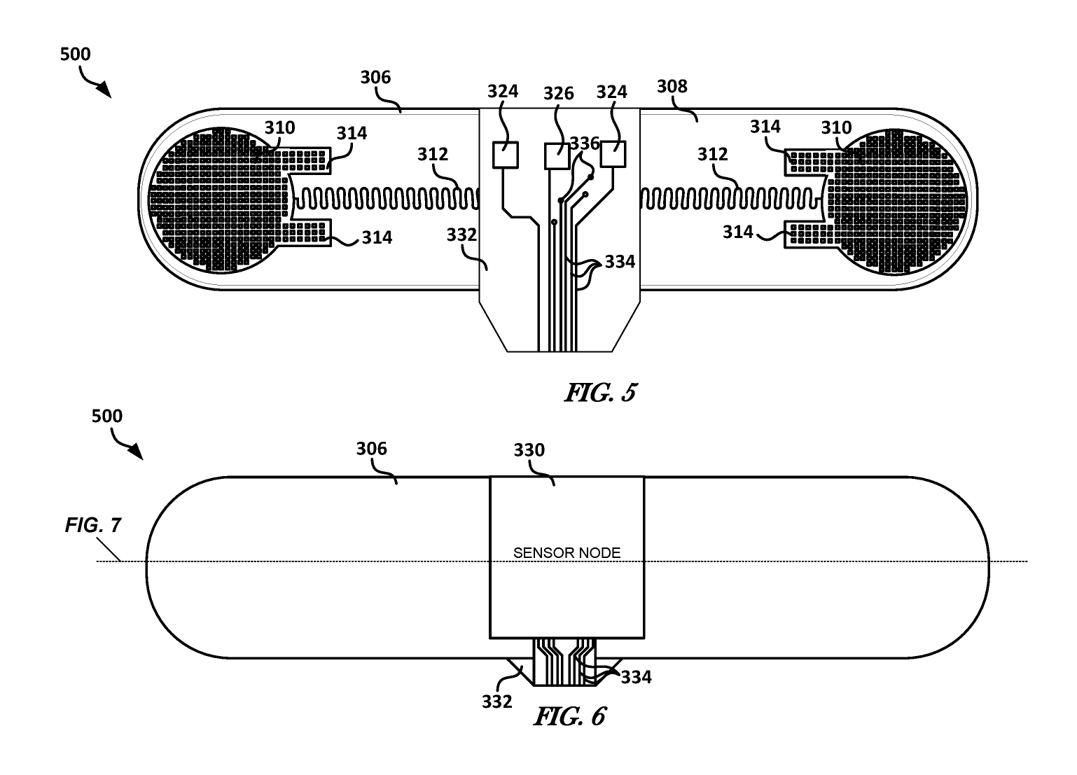

FIG. 5 illustrates, by way of example, a perspective view diagram of an embodiment of another sensor patch system 500 with the flex circuit 332 attached to the stretchable, flexible patch 302. The patch system 500 includes ECG and PPG sensors (e.g., the electrodes 310, the LEDs 324, and the photodiode 326) and does not include the thermistor 328 or the stretch sensor 316. The flex circuit 332 of the system 500 is electrically and mechanically connected to contact pads (e.g., one or more of the contact pads 320) on the substrate 308, such as through a corresponding one or more of the contact pads 338. In embodiments that do not include the stretch sensor 316, the contact pads 320 (see FIG. 3) connected to the stretch sensor 316 may be superfluous or may remain to provide a more secure connection between the flex circuit 332 and the substrate 308. The flex circuit 332 is bent around the fabric 306 and the sensor node 330 is attached to the fabric 306, such as shown in FIG. 6.

The flex circuit 332 as illustrated is attached on two, opposite sides of the patch 302. The flex circuit 332 as illustrated wraps around the patch 302 in a direction generally perpendicular to a longitudinal axis of the patch 302.

FIG. 6 illustrates, by way of example, a perspective view diagram of the embodiment of the sensor patch system 500 of FIG. 5 from an external view. The system 500 includes the sensor node 330 attached to the fabric 306.

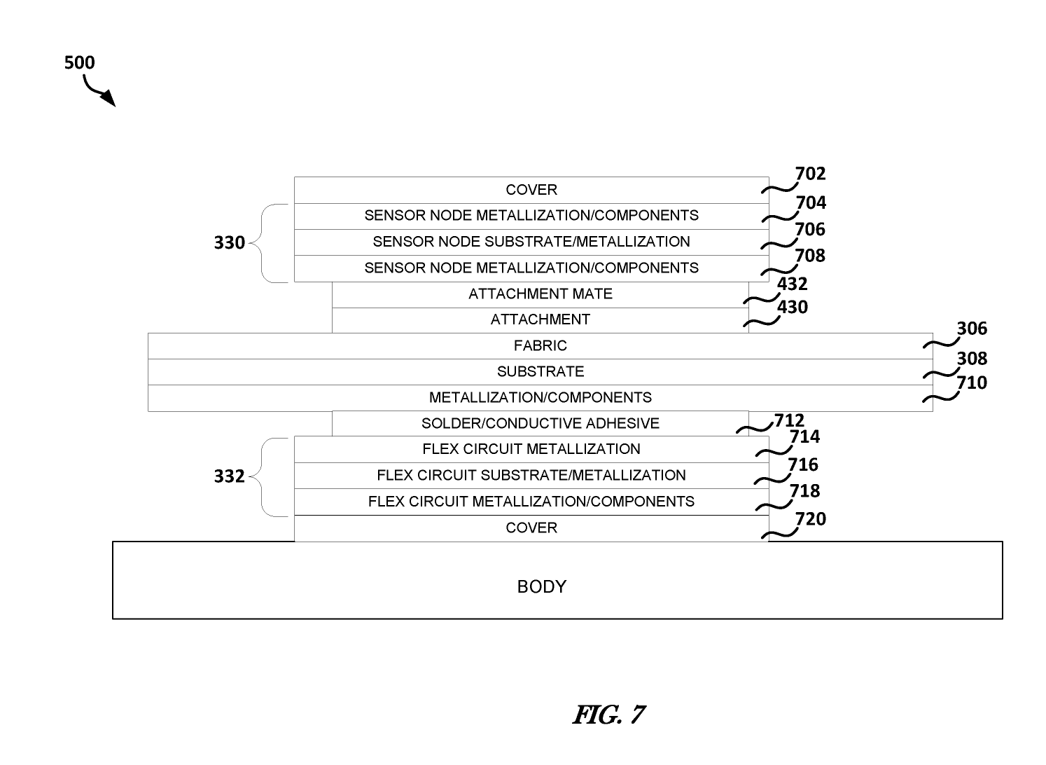

FIG. 7 illustrates, by way of example, a cross-section diagram of the system 500 after the system 500 is cut along the line labelled “FIG. 7” in FIG. 6. The system 500 as illustrated includes an optional cover, sensor node metallization/components 704, sensor node substrate/metallization 706, sensor node metallization/components 708, optional attachment mate 432, optional attachment 430, fabric 306, substrate 308, metallization/components 710, solder/conductive adhesive 712, flex circuit metallization 714, flex circuit substrate/metallization 716, flex circuit metallization/components 718, and another optional cover 720.

The cover 702 can include a material that resists static and/or is water or sweat proof/resistant. In one or more embodiments, the cover 702 includes a plastic, polymer, co-polymer, or other material, such as parylene C. The cover 702 can include a material that protects the sensor node metallization/components 704, sensor node substrate/metallization 706, sensor node metallization/components 708, optional attachment mate 432, optional attachment 430, and/or the fabric 306 from environment external to the system 500. The cover 702, in one or more embodiments includes a polymer, plastic, or other water-impermeable material, such as to protect the components under the 702.

The combination of the sensor node metallization/components 704, sensor node substrate/metallization 706, and sensor node metallization/components 708 form the sensor node 330. In one or more embodiments, the sensor node 330 may include metallization and/or components on only one side and not two as shown in FIG. 7. The sensor node substrate/metallization 706 can include traces, pads, vias, and/or the like in an FR-4, glass, cloth, prepreg, or other substrate material. The components can include components of the sensor node 330, such as is discussed in more detail with regard to FIG. 12.

The metallization/components 710 are the metallization and/or printed components as discussed with regard to FIGS. 3 and 4 (i.e. the first metallization). The solder/conductive adhesive 712 electrically and mechanically connects the flex circuit 332 to the metallization/components 710 that are attached to the substrate 308. The solder/conductive adhesive 712 provides an electrical path for signals from the metallization/components 710 to flow to the flex circuit 332.

The flex circuit 332 comprises the flex circuit metallization 714, flex circuit substrate/metallization 716 and the flex circuit metallization/components 718. The metallization 714 includes one or more pads, vias, and/or traces on the side of the flex circuit facing away from the body, such as the pads 338. The flex circuit substrate/metallization 716 can include traces, pads, vias, and/or the like in a polyamide, or other flexible substrate material. The flex circuit metallization/components 718 include one or more traces, pads, vias, and the components 324, 326, and/or 328.

The optional cover 720 is similar to the cover 702, with the cover 720 protecting metallization and/or components on the opposite side of the system 500 as the cover 702. In one or more embodiments, the cover 720 can include a non-conductive transparent polymer, epoxy, or other material that can protect one or more components of the system 500 from the external environment. In one or more embodiments, the cover 702 is situated so as not be in the optical path of light from the LEDs 724 or in the return path of the light to the photodiode 726. In one or more embodiments, the cover 702 is situated so as to not interfere with a temperature of the thermistor 728.

In one or more embodiments, the cover 720 or the metallization/components 710 includes an adhesive layer thereon, such as to help the device 500 attach too skin of a user. The adhesive layer can include silicone, such as to help enable direct attach to the skin.

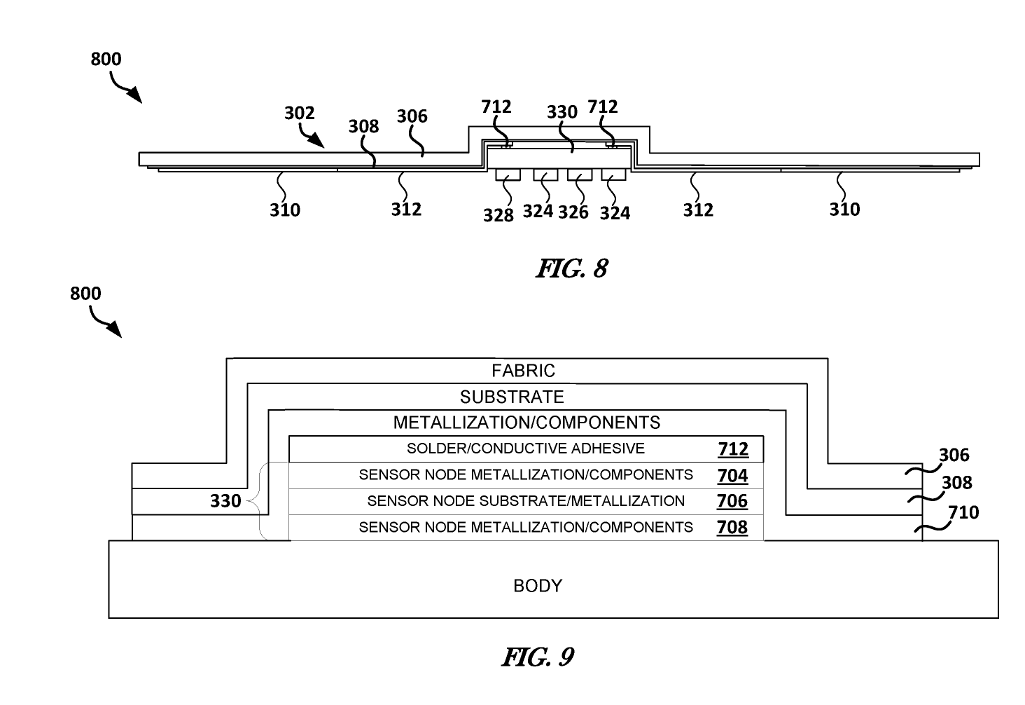

FIG. 8 illustrates, by way of example, a perspective view diagram of an embodiment of another sensor patch system 800 in which the stretchable, flexible patch 302 is situated over the sensor node 330. The sensor node 330, in the embodiment shown in FIG. 8, includes the LEDs 324, the photodiode 326, and the thermistor 328 electrically and mechanically attached thereto. In such an embodiment, the flex circuit 332 may not be needed. However, replacing of the sensor node 330 in the embodiment of FIG. 8 may be more difficult than such replacement in the embodiments of FIGS. 3-6.

FIG. 9 illustrates, by way of example, an exploded view diagram of a stackup of the system 800. The system 800 as illustrated does not include the flex circuit metallization 714, flex circuit substrate/metallization 716 and the flex circuit metallization/components 718, the optional cover 702 and/or 720, the optional attachment mate 432, or the optional attachment 430. The system 800 as illustrated includes the fabric 306, the substrate 308, the metallization/components 710, the sensor node metallization/components 704, sensor node substrate/metallization 706, and sensor node metallization/components 708. The sensor node metallization/components 708 can be in contact with or near the body of the entity wearing the system 800. The solder/conductive adhesive 712 electrically and mechanically connects the metallization/components 710 to the sensor node 330, thus connecting the flexible, stretchable patch to the sensor node 330.

FIG. 10 illustrates, by way of example, a cross-section diagram of an embodiment of another sensor patch system 1000 in which the sensors contact or be near the skin through one or more holes in the stretchable flexible patch. In the embodiment shown in FIG. 10, the fabric 306 is optional. The patch system 1000 as illustrated includes a hole 1006 in the substrate 308 and fabric 306 through which the sensor node 330 and corresponding sensors 324, 326, and 328 can access the body of the user. The sensor node 330 can include a pin, snap, or other connection feature 1002 that can be attached to a mating connection feature 1004 on, or at least partially in, the fabric 306 or substrate 308.

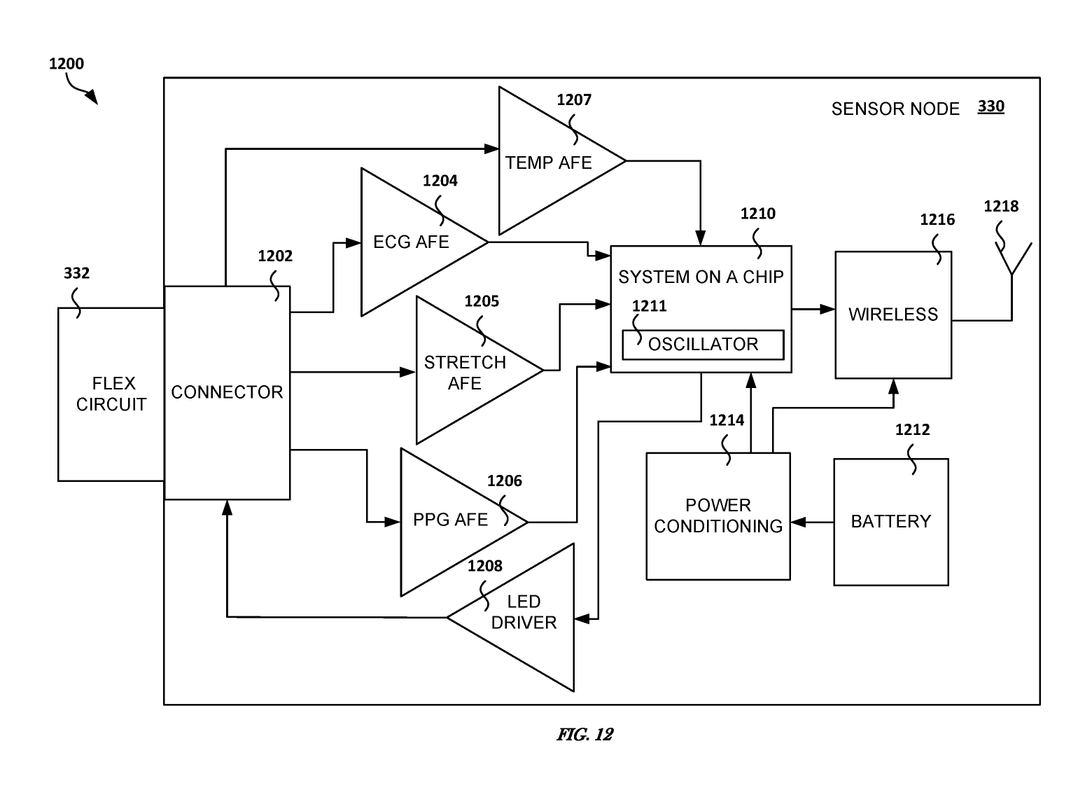

FIG. 11 illustrates, by way of example, an exploded view diagram of a stackup of the system 1000. The system 1000 as illustrated includes the optional fabric 306. In embodiments that do not include the fabric 306, the connection feature 1004 can be attached to the substrate 308. The system 800 as illustrated includes the fabric 306, the substrate 308, the metallization/components 710, the sensor node metallization/components 704, sensor node substrate/metallization 706, and sensor node metallization/components 708. The sensor node metallization/components 708 and/or the metallization/components 710 can be in contact with or near the body of the entity wearing the system 1000. The connection features 1002 and 1004 can connect the sensor node 330 to the flexible patch.

FIG. 12 illustrates, by way of example, a logical circuit diagram of sensor circuitry 1200, such as can be included on one or more of the sensor node 330, the flex circuit 332, and/or the substrate 308. The sensor circuitry 1200 as illustrated includes the flex circuit 332 and the sensor node 330. Note that in embodiments in which the flex circuit 332 is not used, the sensor node 330 can receive signals directly from metallization on the substrate 308 (see FIGS. 8-11, for example). In one or more embodiments, one or more of the components of the circuitry 1200 can be connected to the substrate 306 rather than the sensor node 330.

The flex circuit 332 is connected to the sensor node 330, such as through a connector 1202. The connector 1202 includes conductive contacts that, when exposed portions of conductors (e.g., metallization) on the flex circuit 332 are in contact therewith, form an electrical path for electrical signals from the flex circuit 332. The connector 1202 also includes a releasable attachment mechanism that secures the flex circuit 332 to the sensor node 330. Using the connector 1202, instead of hardwiring or soldering the flex circuit 332 to the sensor node 330, allows the sensor node 330 (and possibly even the flex circuit 332 depending on the electrical and/or mechanical connections between the flex circuit 332 and the substrate 308) to be reusable. Such as design also allows for the flexible portions (e.g., the flex circuit 332, the fabric 306, and everything thereon) to be disposable. Such embodiments can help save money by only needing to replace the flexible portion (the parts of the system in contact with the human body) and not the sensor node 330. The connector 1202 provides the ability to mechanically/electrically connect and disconnect the sensor node 330 from the flex circuit 332.

Signals from the flex circuit 332 or the metallization or components on the substrate 308 are routed (e.g., through the connector 1202) to an ECG analog front end (AFE) 1204 a PPG AFE 1206, a stretch AFE 1205, or a sensor AFE 1207 (e.g., a temperature and/or a blood oxygen saturation AFE). The ECG AFE 1204 receives signals from the electrodes 310 and the meandering traces 312, such as through metallization of the flex circuit 332 or through a direct electrical connection between the sensor node 330 and the metallization on the substrate 308 (e.g., a direct connection to the pad 320). The stretch AFE 1205 receives signals from the stretch sensor 316, such as through the flex circuit 332 or through a direct electrical connection between the sensor node 330 and the metallization on the substrate 308. The PPG AFE 1206 receives signals from the photodiode 326, such as through the flex circuit 332 or through a direct electrical connection between the sensor node 330 and the metallization on the substrate 308. The temperature AFE 1207 receives signals from the sensor 328, such as through the flex circuit 332 or through a direct electrical connection between the sensor node 330 and the metallization on the substrate 308.

Each of the AFEs (e.g., the ECG AFE 1204, the stretch AFE 1205, the PPG AFE 1206, and the temperature AFE 1207) are optional. If the application includes a corresponding sensor, the sensor node 330 can include the corresponding AFE. For example, an application that includes sensing of only ECG and PPG to monitor BP, may not include the stretch AFE 1205 or the temp AFE 1207, but may include the ECG AFE 1204, the PPG AFE 1206 (and an LED driver 1208). In another example, a respiration and temperature sensor system may include the temp AFE 1207, the stretch AFE 1205, and may not include the ECG AFE 1204 and the PPG AFE 1206 (and the LED driver 1208).

Each of the AFEs (e.g., the ECG AFE 1204, the stretch AFE 1205, the PPG AFE 1206, and the temperature AFE 1207) include circuitry specific to a sensing application, such as to sense, amplify, and/or condition the signals from the respective sensor (e.g., the electrodes 310, stretch sensor 316, photodiode 326, or thermistor 328). Such circuitry can include analog and/or digital circuitry, such as can include one or more transistors, resistors, capacitors, inductors, diodes, amplifiers, analog to digital converters (ADC), high-pass, low-pass, sensors or other circuitry for measuring motion, noise, other bio-signals, band pass filters, or the like. For example, the circuitry can include an AFE for sensing electrode impedance (between ECG electrodes) and to generate a noise signal that can be used for motion noise cancellation from ECG signals. The circuitry can additionally or alternatively include a stretch sensor (similar to the stretch sensor 316) to help measure lateral forces on the patch. Such measurements can help in cancelling other noise from the patch. The circuitry can include an inertial measurement unit (IMU) that can be used to track motion (in one, two, or three directions). Such measurements can be used to help cancel motion artifacts in bio-signals and additionally or alternatively for general motion tracking.

The signals from the AFEs can be provided to a system on a chip (SOC) 1210. The SOC 1210 performs operations on the signals from the AFEs, such as can include, providing a timestamp with the signals, digitizing the signals (e.g., using an ADC), temporally correlating signals, determining a measurement based on the signals (e.g., BP, temperature, HR, respiration rate, or the like), removing noise from bio-signals (or other signals), such as by using digital signal processing (DSP) techniques, adaptive noise cancellation based on provides signals indicative of noise, user motion, and/or activity tracking, storing signals for later retrieval and/or processing, and/or identifying fiducial point(s) or features in the bio-signals or other signals, such as R-wave peak in ECG signals, a peak of a PPG signal, such as to help calculate PTT, such as can be used for blood pressure estimation. Note that in one or more embodiments, the SOC 1210 does not perform some operations listed and such operations can be performed by an application of the mobile device 106. For example, in one or more embodiments, the SOC 1210 can timestamp or temporally correlate data and provide the data to the mobile device 106 (e.g., through a wireless transmitter 1216 and antenna 1218). The mobile device 106 can then use those data signals to determine the BP, HR, respiration, temperature, or other bio-measurement. Such a configuration can use less power on the sensor node 330 at the expense of more resource usage at the mobile device 106.

The SOC 1210 can be driven by a single oscillator 1211. Using a single oscillator 1211 can help time-synchronize signals that have time-synchronization requirements, such as BP signals (e.g., the ECG and PPG signals). To get an accurate measure of BP using ECG and PPG signals, the signals from the ECG and PPG sensors should be correlated to within a sub-second. Such correlation provides an ability to accurately determine the PPT and ultimately the BP accurately. Using only the single oscillator 1211 provides an advantage over the prior systems that includes separate oscillators for the PPG and ECG signal paths. Such systems either do not provide an accurate measure of the BP due to insufficient temporal correlation between the ECG and PPG signals, or include circuitry to perform the temporal correlation, which increases the power draw of the circuit and the overall complexity of the system.

The sensor node 330 as illustrated includes a battery 1212 to provide power to the SOC and/or wireless transmission circuitry 1216. The battery 1212, in one or more embodiments, is replaceable and/or rechargeable. In one or more embodiments, the power from the battery 1212 is provided to power conditioning circuitry 1214. The power conditioning circuitry 1214 can include one or more voltage or current regulators, one or more voltage or current boosters, or other the like, to alter a voltage or current level provided by the battery 1212. For example, the different AFEs may operate at different voltage levels and may require different voltage or current rails for proper digitization of signals, the LED driver 1208 may operate at a different voltage or current level than the SOC 1210 or one or more of the AFEs, or the wireless transmission circuitry 1216 may operate at a different voltage or current level than other circuitry of the sensor node 330 or the sensors 324, 326, or 328. Note that power from the battery 1212 and/or the power conditioning circuitry 1214 may be provided to one or more of the AFEs, the LED driver 1208, the photodiode 326, or the thermistor 328 as an application may require. Such connections are not shown in the FIG. 12 such as to help not obscure the view of the circuitry in FIG. 12.

The wireless transmission circuitry 1216 includes a transmission radio to provide modulated radio signals to an antenna 1218. The antenna 1218, when excited by the radio signals from the transmission circuitry 1216, radiates electromagnetic waves. The combination of the transmission circuitry 1216 and the antenna 1218 can provide signals to the mobile device 106. The signals can be Bluetooth, induction wireless, infrared wireless, ultra-wideband, ZigBee, or other personal area network compliant signals. In one or more embodiments, the SOC 1210 includes a memory on which signal data from the AFEs is stored. This signal data can be transmitted (via the wireless transmission circuitry 1216 and antenna 1218) in response to a device, such as the mobile device 106, coming to within sufficient range of the sensor node 330. Such an embodiment can help loosen a requirement that the patch systems be within range of a mobile device to provide data, such as for monitoring or other analysis. For example, in an embodiment in which the wireless transmission circuitry 1216 and the antenna 1218 are configured for near field communication, the memory can store data from the AFEs until the mobile device 106 is within about four centimeters or less of the sensor node 330. In response to detecting the mobile device 106 is within range, the SOC 1210 can dump the contents of the memory to the mobile device 106.

In one or more embodiments, the sensor node 330 does not include the wireless transmission circuitry 1216 or the antenna 1218. In such embodiments, the sensor node 330 can include a wired communications port through which the data from the memory can be transferred. The wired communications port can operate in accord with a universal serial bus (USB), Ethernet, RS232, peripheral component interconnect (PCI), lightning, firewire, or other wired serial or parallel communication protocol.

In one or more embodiments, the SOC includes a speaker or motor that can provide audible or tactile indications to the entity wearing the patch system. In one or more embodiments, the speaker or motor can indicate when the memory is full and should be dumped to the mobile device 106, when the patch system is connected to the mobile device 106, when the power available from the battery 1212 is below a specified threshold, if a signal is not being received from a component, such as the stretch sensor 316, LEDs 324, photodiode 326, thermistor 328, electrodes 310, meandering traces 312, or other component, if the flex circuit 332 or the sensor node 330, or the patch 302 should be replaced, among others.

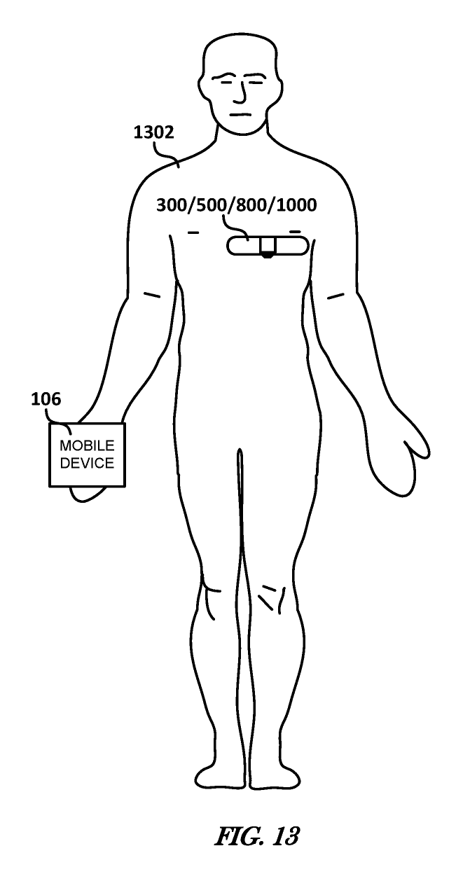

FIG. 13 illustrates, by way of example, a perspective view diagram of an embodiment of an entity 1302 wearing the sensor patch system 300, 500, 800, or 1000. The orientation of the sensor patch system 300, 500, 800, or 1000 is merely an example, and other orientations are possible and may even have stronger bio-signal detection. The fabric 306 of the patch system 300, 500, 800, or 1000 can be attached to the skin using a bio-compatible adhesive film (e.g., a silicon adhesive sheet, spray, or other attachment mechanism. For example, a male connection portion of a button, Velcro, or other attachment mechanism can be adhered to the entity 1302 and the patch system 300, 500, 800, or 1000 can include a mating connection portion that is attached to the attachment mechanism adhered to the user's skin. The attachment mechanism can be situated on the body side of the patch (i.e. the side shown in FIG. 3).

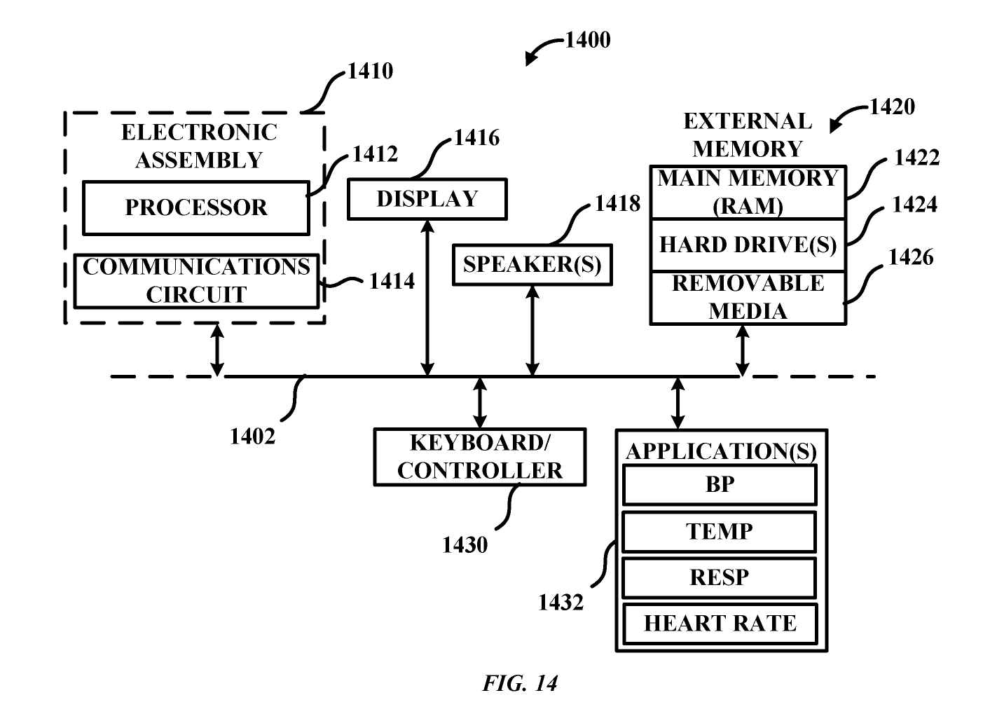

FIG. 14 illustrates, by way of example, a block diagram of an embodiment of an electronic device 1400. The electronic device 1400 includes components which can be a part of the mobile device 106, the sensor node 330, or the flex circuit 332, or the metallization or components on the substrate 306, for example. The electronic device 1400 is merely an example of a device in which embodiments of the present disclosure can be used.

In the example of FIG. 14, electronic device 1400 comprises a data processing system that includes a system bus 1402 to couple the various components of the system. System bus 1402 provides communications links among the various components of the electronic device 1400 and can be implemented as a single bus, as a combination of busses, or in any other suitable manner.

An electronic assembly 1410 is coupled to system bus 1402. The electronic assembly 1410 can include a circuit or combination of circuits. In one embodiment, the electronic assembly 1410 includes a processor 1412 which can be of any type. As used herein, “processor” means any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, or any other type of processor or processing circuit.

Other types of circuits that can be included in electronic assembly 1410 are a custom circuit, an application-specific integrated circuit (ASIC), or the like, such as, for example, one or more circuits (such as a communications circuitry 1414) for use in wireless devices like mobile telephones, pagers, personal data assistants, portable computers, two-way radios, and similar electronic systems. The electronic assemble can perform any other type of function, such as can be requested by application(s) 1432. The communications circuitry 1414 can include wired or wireless communications circuitry capable of communicating with the wired or wireless transmission circuitry of the patch system 300, 500, 800, or 1000. The communications circuitry 1414 can include one or more transceivers, antennas, modulators/demodulators, amplifiers, decoders, or other circuitry for communicating with the mobile device 106.

The electronic device 1400 can include an external memory 1420, which in turn can include one or more memory elements suitable to the particular application, such as a main memory 1422 in the form of random access memory (RAM), one or more hard drives 1424, and/or one or more drives that handle removable media 1426 such as compact disks (CD), digital video disk (DVD), and the like.

The electronic device 1400 can also include a display device 1416, one or more speakers 1418, and a keyboard and/or controller 1430, which can include a mouse, trackball, touch screen, voice-recognition device, or any other device that permits a system user to input information into and receive information from the electronic device 1400.

The electronic device 1400 can include one or more application(s) 1432 executing thereon. The application(s) as illustrated include a BP, temperature, respiration, and heart rate application. The BP application can receive ECG, PPG, and/or PPT data from the patch system 300, 500, 800, or 1000 and produce estimates of the blood pressure based on the provided data. The temperature application can receive data from the thermistor 328 in digitized form (e.g., digitized by the SOC 1210) and determine a temperature based on the digitized thermistor 328 data. The respiration application can receive data from the stretch sensor 316 in digitized form and provide data indicating a rate and/or depth of breathing of the entity wearing the system 300, 500, 800, or 1000. The heart rate application can receive digitized forms of the data from the electrodes 310 and determine a heart rate based on the ECG data.

### Additional Notes and Examples

In Example 1 a device includes a stretchable, flexible substrate material mechanically coupled to and on the stretchable, flexible fabric and first metallization mechanically coupled to and on the substrate material, the first metallization including first pads, meandering traces, and at least one of electrocardiogram (ECG) electrodes, a stretch sensor, and at least one stretch limiting patch.

In Example 2 the device of Example 1 can include, a stretchable, flexible fabric and wherein the stretchable flexible substrate material is mechanically coupled to and on the stretchable, flexible fabric.

In Example 3 the device of at least one of Examples 1-2 can include a sensor node electrically coupled to the first pads, the sensor node including circuitry to amplify and digitize signals transmitted through the pads, the sensor node including communications circuitry to wired or wirelessly communicate digitized signals to an external device.

In Example 4 the device of at least one of Examples 1-3, can include a flex circuit including second metallization therein and thereon, the second metallization including second traces and second pads, the second pads respectively electrically and mechanically connected to the first pads by one of a solder and a conductive adhesive and a connector electrically and mechanically connected to the sensor node, the flex circuit electrically and mechanically connected to the connector to electrically couple signals from the first metallization to the sensor node.

In Example 5 the device of Example 4 can include at least one sensor electrically connected to a pad of the second pads, the at least one sensor including at least one of a photodiode and a resistance temperature detector (RTD).

In Example 6 the device of at least one of Examples 4-5 can include at least one light emitting diode (LED) electrically connected to another pad of the second pads.

In Example 7 the device of at least one of Examples 3-6 can include, wherein the sensor node includes only one oscillator and the circuitry temporally correlates data from the photodiode with data from the ECG electrodes.

In Example 8 the device of at least one of Examples 4-7 can include at least one sensor electrically connected to a pad of the second pads, the at least one sensor including at least one of a photodiode and a resistance temperature detector (RTD).

In Example 9 the device of at least one of Examples 4-8 can include at least one light emitting diode (LED) electrically connected to another pad of the second pads.

In Example 10 the device of at least one of Examples 3-9 can include, wherein the sensor node includes only one oscillator and the circuitry temporally correlates data from the photodiode with data from the ECG electrodes.

In Example 11 the device of at least one of Examples 1-10 can include, wherein the substrate includes an elastomer material.

In Example 12 the device of at least one of Examples 1-11 can include, wherein the ECG electrodes include a plurality of holes therethrough.

In Example 13 the device of at least one of Examples 1-12 can include an adhesive on the first metallization to help in attaching the device to skin of a user.

In Example 14 the device of at least one of Examples 1-13 can include, wherein the stretch limiting patch includes a solid patch of conductive material.

In Example 15 the device of Example 14 can include, wherein the solid patch of conductive material includes a plurality of holes therethrough.

In Example 16 the device of at least one of Examples 14-15 can include, wherein the patch of conductive material is substantially rectangular.

In Example 17 the device of at least one of Examples 1-16 can include, wherein the stretch limiting patch is one of a plurality of stretch limiting patches mechanically coupled to and on the substrate, the stretch limiting patches situated so as to reduce stretchability of the fabric and the substrate material at and near junctions of the meandering traces and the ECG electrodes.

In Example 18 the device of Example 17 can include, wherein the stretch limiting patches are situated so as to reduce stretchability of the fabric and the substrate material at and near junctions of the meandering traces and the first pads.

In Example 19 the device of at least one of Examples 1-18 can include, wherein the stretch sensor includes at least two sets of meandering lines electrically connected to each other and to at least two pads of the first pads.

In Example 20 the device of Example 19 can include, wherein the stretch sensor forms an open loop.

In Example 21 a system includes a stretchable, flexible patch comprising a stretchable, flexible fabric, a stretchable, flexible substrate material mechanically coupled to and on the stretchable, flexible fabric, and first metallization mechanically coupled to and on the substrate material, the first metallization including first pads, meandering traces, and at least one of electrocardiogram (ECG) electrodes, a stretch sensor, and at least one stretch limiting patch, a sensor node electrically coupled to the first pads, the sensor node comprising circuitry to amplify and digitize signals transmitted through the pads, and communications circuitry to wired or wirelessly communicate digitized signals from the circuitry, and a mobile device to receive the digitized signals from the communications circuitry and determine a measure of a biological function of an entity based on the digitized signals.

In Example 22 the system of Example 21 can include a flex circuit including second metallization therein and thereon, the second metallization including traces and second pads, the second pads electrically and mechanically connected to the first pads by one of a solder and a conductive adhesive, and a connector electrically and mechanically connected to the sensor node, the flex circuit electrically and mechanically connected to the connector to electrically couple signals from the first metallization.

In Example 23 the system of Example 22 can include at least one sensor electrically connected to a pad of the second pads, the at least one sensor including at least one of a photodiode and a resistance temperature detector (RTD).

In Example 24 the system of at least one of Examples 21-23 can include at least one light emitting diode (LED) electrically connected to another pad of the second pads.

In Example 25 the system of at least one of Example 24 can include, wherein the sensor node includes only one oscillator and the circuitry temporally correlates data from the photodiode with data from the ECG electrodes.

In Example 26 the system of at least one of Examples 21-25 can include, at least one sensor electrically connected to a pad of the second pads, the at least one sensor including at least one of a photodiode and a resistance temperature detector (RTD).

In Example 27 the system of Example 26 can include at least one light emitting diode (LED) electrically connected to another pad of the second pads.

In Example 28 the system of at least one of Examples 21-27 can include, circuitry to detect at least one of noise and motion, and other circuitry to cancel noise based on signals detected by the circuitry.

In Example 29 the system of at least one of Examples 21-28 can include, wherein the sensor node includes only one oscillator and the circuitry is to temporally correlate data from the photodiode with data from the ECG electrodes.

In Example 30 the system of at least one of Examples 21-29 can include, wherein the substrate includes thermo-plastic polyurethane (TPU).

In Example 31 the system of at least one of Examples 21-30 can include, wherein the ECG electrodes include a plurality of holes therethrough.

In Example 32 the system of at least one of Examples 21-31 can include, wherein the stretch limiting patch includes a solid patch of conductive material.

In Example 33 the system of Example 32 can include, wherein the solid patch of conductive material includes a plurality of holes therethrough.

In Example 34 the system of at least one of Examples 32-33 can include, wherein the patch of conductive material is substantially rectangular.

In Example 35 the system of at least one of Examples 21-34 can include, wherein the stretch limiting patch is one of a plurality of stretch limiting patches mechanically coupled to and on the substrate, the stretch limiting patches situated so as to reduce stretchability of the fabric and the substrate material at and near junctions of the meandering traces and the ECG electrodes.

In Example 36 the system of Example 35 can include, wherein the stretch limiting patches are situated so as to reduce stretchability of the fabric and the substrate material at and near junctions of the meandering traces and the first pads.

In Example 37 the system of at least one of Examples 21-36 can include, wherein the stretch sensor includes at least two sets of meandering lines electrically connected to each other and to at least two pads of the first pads.

In Example 38 the system of Example 37 can include, wherein the stretch sensor forms an open loop.

In Example 39 a device can include a stretchable, flexible patch including a stretchable, flexible fabric, a stretchable, flexible substrate material mechanically coupled to and on the stretchable, flexible fabric, and first metallization mechanically coupled to and on the substrate material, the first metallization including first pads, meandering traces electrically connected to the first pads, and at least one of electrocardiogram (ECG) electrodes, a stretch sensor, and at least one stretch resist patch, a sensor node electrically coupled to the first pads, the sensor node including circuitry to digitize signals received from the patch, a connector, electrically and mechanically connected to second metallization on the sensor node, and communications circuitry to wired or wirelessly communicate the digitized signals to an external device, and a flex circuit including a polyamide substrate, and second metallization at least partially in the polyamide substrate, the second metallization including traces and second pads, the second pads electrically and mechanically connected to the first pads by one of a solder and a conductive adhesive, the second metallization including traces electrically and mechanically connected to the connector to electrically couple signals from the first metallization to the sensor node through the flex circuit.

In Example 40 the device of Example 39 includes at least one sensor electrically connected to a pad of the second pads, the at least one sensor including at least one of a photodiode and a resistance temperature detector (RTD).

In Example 41 the device of at least one of Examples 39-40 includes at least one light emitting diode (LED) electrically connected to another pad of the second pads.

In Example 42 the device of at least one of Examples 39-41 can include, wherein the sensor node includes only one oscillator and the circuitry temporally correlates data from the photodiode with data from the ECG electrodes.

In Example 43 the device of at least one of Examples 39-42 can include, wherein the ECG electrodes are dry ECG electrodes that include a solid patch of conductive material with a plurality of perforations therethrough.

In Example 44 the device of at least one of Examples 39-43 can include, wherein the substrate includes thermo-plastic polyurethane (TPU).

In Example 45 the device of at least one of Examples 39-44 can include, wherein the stretch limiting patch includes a solid patch of conductive material.

In Example 46 the device of Example 45 can include, wherein the solid patch of conductive material includes a plurality of holes therethrough.

In Example 47 the device of at least one of Examples 45-46 can include, wherein the patch of conductive material is substantially rectangular.

In Example 48 the device of at least one of Examples 45-47 can include, wherein the stretch limiting patch is one of a plurality of stretch limiting patches mechanically coupled to and on the substrate, the stretch limiting patches situated so as to reduce stretchability of the fabric and the substrate material at and near junctions of the meandering traces and the ECG electrodes.

In Example 49 the device of at least one of Example 48 can include, wherein the stretch limiting patches situated so as to reduce stretchability of the fabric and the substrate material at and near junctions of the meandering traces and the first pads.

In Example 50 the device of at least one of Examples 39-49 can include, wherein the stretch sensor includes at least two sets of meandering lines electrically connected to each other and to at least two pads of the first pads.

In Example 51 the device of Example 50 can include, wherein the stretch sensor forms an open loop.

The above description of embodiments includes references to the accompanying drawings, which form a part of the description of embodiments. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above description of embodiments, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the description of embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-20. (canceled)

21. A device comprising:
a stretchable, flexible first substrate material mechanically coupled to and on the stretchable, flexible fabric; and
first metallization mechanically coupled to and on the first substrate material, the first metallization including first pads, meandering traces, and at least one of electrocardiogram (ECG) electrodes, a stretch sensor, and at least one stretch limiting patch.

22. The device of claim 21, further comprising:
a stretchable, flexible fabric; and
wherein the stretchable flexible first substrate material is mechanically coupled to and on the stretchable, flexible fabric.

23. The device of claim 21, further comprising:
a sensor node electrically coupled to the first pads, the sensor node including circuitry to amplify and digitize signals transmitted through the pads, the sensor node including communications circuitry to wired or wirelessly communicate digitized signals to an external device.

24. The device of claim 23, further comprising:
a flex circuit including second metallization therein and thereon, the second metallization including second traces and second pads, the second pads respectively electrically and mechanically connected to the first pads by one of a solder and a conductive adhesive; and
a connector electrically and mechanically connected to the sensor node, the flex circuit electrically and mechanically connected to the connector to electrically couple signals from the first metallization to the sensor node.

25. The device of claim 24, further comprising:
at least one sensor electrically connected to a pad of the second pads, the at least one sensor including at least one of a photodiode and a resistance temperature detector (RTD).

26. The device of claim 25, further comprising:
at least one light emitting diode (LED) electrically connected to another pad of the second pads.

27. The device of claim 26, wherein the sensor node includes only one oscillator and the circuitry temporally correlates data from the photodiode with data from the ECG electrodes.

28. The device of claim 23, further comprising:
at least one sensor electrically connected to a pad of the sensor node, the at least one sensor including at least one of a photodiode and a resistance temperature detector (RTD).

29. The device of claim 28, further comprising:
at least one light emitting diode (LED) electrically connected to another pad of the second pads.

30. The device of claim 29, wherein the sensor node includes only one oscillator and the circuitry temporally correlates data from the photodiode with data from the ECG electrodes.

31. A system comprising:
a stretchable, flexible patch comprising:
a stretchable, flexible fabric;
a stretchable, flexible substrate material mechanically coupled to and on the stretchable, flexible fabric; and
first metallization mechanically coupled to and on the substrate material, the first metallization including first pads, meandering traces, and at least one of electrocardiogram (ECG) electrodes, a stretch sensor, and at least one stretch limiting patch;
a sensor node electrically coupled to the first pads, the sensor node comprising:
circuitry to amplify and digitize signals transmitted through the pads; and
communications circuitry to wired or wirelessly communicate digitized signals from the circuitry; and
a mobile device to receive the digitized signals from the communications circuitry and determine a measure of a biological function of an entity based on the digitized signals.

32. The system of claim 31, further comprising:
a flex circuit including second metallization therein and thereon, the second metallization including traces and second pads, the second pads electrically and mechanically connected to the first pads by one of a solder and a conductive adhesive; and
a connector electrically and mechanically connected to the sensor node; the flex circuit electrically and mechanically connected to the connector to electrically couple signals from the first metallization.

33. The device of claim 32, further comprising:
at least one sensor electrically connected to a pad of the second pads, the at least one sensor including at least one of a photodiode and a resistance temperature detector (RTD).

34. The device of claim 33, further comprising:
at least one light emitting diode (LED) electrically connected to another pad of the second pads.

35. The device of claim 34, wherein the sensor node includes only one oscillator and the circuitry temporally correlates data from the photodiode with data from the ECG electrodes.

36. A device comprising:
a stretchable, flexible patch comprising:
a stretchable, flexible fabric;
a stretchable, flexible substrate material mechanically coupled to and on the stretchable, flexible fabric; and
first metallization mechanically coupled to and on the substrate material, the first metallization including first pads, meandering traces electrically connected to the first pads, and at least one of electrocardiogram (ECG) electrodes, a stretch sensor, and at least one stretch resist patch;
a sensor node electrically coupled to the first pads, the sensor node comprising:
circuitry to digitize signals received from the patch;
a connector, electrically and mechanically connected to second metallization on the sensor node; and
communications circuitry to wired or wirelessly communicate the digitized signals to an external device; and
a flex circuit comprising:
a polyamide substrate; and
third metallization at least partially in the polyamide substrate, the third metallization including traces and second pads, the second pads electrically and mechanically connected to the first pads by one of a solder and a conductive adhesive, the third metallization including traces electrically and mechanically connected to the connector to electrically couple signals from the first metallization to the sensor node through the flex circuit.

37. The device of claim 36, further comprising:
at least one sensor electrically connected to a pad of the second pads, the at least one sensor including at least one of a photodiode and a resistance temperature detector (RTD).

38. The device of claim 37, further comprising:
at least one light emitting diode (LED) electrically connected to another pad of the second pads.

39. The device of claim 38, wherein the sensor node includes only one oscillator and the circuitry temporally correlates data from the photodiode with data from the ECG electrodes.

40. The device of claim 36, wherein the ECG electrodes are dry ECG electrodes that include a solid patch of conductive material with a plurality of perforations therethrough.
